(19) United States
(12) Patent Application Publication
(10) Pub. No.: US 2006/0204547 A1
(43) Pub. Date: Sep. 14, 2006

(54) Drug delivery stent with extended in vivo release of anti-inflammatory

(75) Inventors: Thai Minh Nguyen, Santa Clara, CA (US); John F. Shanley, Redwood City, CA (US); Frank Litvack, Los Angeles, CA (US); Theodore L. Parker, Danville, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/375,595

(22) Filed: Mar. 14, 2006

Related U.S. Application Data
(60) Provisional application No. 60/662,040, filed on Mar. 14, 2005.

(51) Int. Cl. A61K 31/4745; A61F 2/06

(52) U.S. Cl. 424/426; 514/291; 623/1.11

(57) ABSTRACT

A method for decreasing the level of restenosis following a stent placement medical intervention involves the continuous administration of a dose of an anti-restenotic agent, such as Pimecrolimus, from the stent to vascular tissue in need of treatment in a controlled and extended drug release profile for a period of at least 45 days in vivo. The in vivo release profile is determined by in vivo animal experiments involving implanting a series of stents in animals, explanting the stents from the animals at selected time points, and extracting remaining drug from the explanted stents.

### CROSS REFERENCE TO RELATED APPLICATIONS

This application claims priority to U.S. Provisional Patent Application Ser. No. 60/662,040, filed Mar. 14, 2005, the entire contents of which are incorporated herein by reference.

### BACKGROUND

Most coronary artery-related deaths are caused by atherosclerotic lesions which limit or obstruct coronary blood flow to heart tissue. To address coronary artery disease, doctors often resort to percutaneous transluminal coronary angioplasty (PTCA) or coronary artery bypass graft (CABG). PTCA is a procedure in which a small balloon catheter is passed down a narrowed coronary artery and then expanded to re-open the artery. The major advantage of angioplasty is that patients in which the procedure is successful need not undergo the more invasive surgical procedure of coronary artery bypass graft. A major difficulty with PTCA is the problem of post-angioplasty closure of the vessel, both immediately after PTCA (acute reocclusion) and in the long term (restenosis).

Coronary stents are typically used in combination with PTCA to reduce reocclusion of the artery. Stents are introduced percutaneously, and transported transluminally until positioned at a desired location. The stents are then expanded either mechanically, such as by the expansion of a mandrel or balloon positioned inside the stent, or expand themselves by releasing stored energy upon actuation within the body. Once expanded within the lumen stents become encapsulated within the body tissue and remain a permanent implant.

Restenosis is a major complication that can arise following vascular interventions such as angioplasty and the implantation of stents. Simply defined, restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen. To treat restenosis, additional revascularization procedures are frequently required, thereby increasing trauma and risk to the patient.

While the exact mechanisms of restenosis are still being determined, certain agents have been demonstrated to reduce restenosis in humans. Drug eluting stents represent the most advanced and sophisticated treatment currently available to address restenosis. Two examples of agents which have been demonstrated to reduce restenosis when delivered from a stent are paclitaxel, a well-known compound that is commonly used in the treatment of cancerous tumors, and Rapamycin, an immunosuppressive compound used to prevent rejection of organ or tissue transplants.

Currently marketed drug-eluting stents are bare metal stents that are coated on the surface with a drug and a biostable polymer to reduce restenosis by inhibiting the growth or proliferation of neointima. In addition to polymer coated stents other polymer and non-polymer drug delivery systems are in development to allow delivery of antiproliferative drugs from stents.

Drug eluting stent systems are tested in various in vitro test systems to determine the kinetic release profile, also called the release kinetics, or amount of drug released from the polymer system over time. Clinical trials have demonstrated that a drug's release kinetics in addition to total dose have an effect on clinical outcomes. The in vitro test processes generally include placing a stent into an artificial release medium for a period of time, removing the stent from the release medium, and analyzing the release medium, such as by HPLC, to determine the amount of drug released from the stent during that period. This procedure is repeated at a number of time points and the cumulative drug release is plotted vs. time as a release kinetic profile. It has been shown that the release kinetic from the in vitro analysis can vary significantly depending on the release medium and test procedure used. Further it is difficult to compare different polymer/drug systems in an in vitro model since different polymers and drugs respond differently to the same release medium. In vitro release kinetics are seldom reflective of the in vivo release within an actual artery.

Thus, it would be desirable be able to characterize a release kinetic of a drug eluting stent based on in vivo data in an animal model which provides a close correlation to the human body.

### SUMMARY OF THE INVENTION

The present invention relates to methods of reducing restenosis and stents for reducing restenosis which deliver drug in vivo over an extended administration period of at least 45 days.

In accordance with one aspect of the invention, a method of reducing restenosis comprises the steps of providing a drug delivery stent having a dosage of Pimecrolimus for delivery to an artery, implanting the stent within an artery of a patient, and delivering Pimecrolimus from the stent in vivo over an administration period beginning on the date of implantation and ending between 45 days and 1 year after implantation.

In accordance with another aspect of the invention, a stent for reducing restenosis is comprised of a drug delivery stent having initial unexpanded diameter for insertion of the stent into a coronary artery and an expanded diameter for implantation within a coronary artery, the stent having a dosage of Pimecrolimus for delivery to an artery, wherein the dosage of Pimecrolimus is arranged to be released over an administration period beginning on the date of implantation and ending between 45 days and 1 year after implantation.

In accordance with an additional aspect of the invention, a stent for reducing restenosis is comprised of a drug delivery stent having initial unexpanded diameter for insertion of the stent into a coronary artery and an expanded diameter for implantation within a coronary artery, the stent having a dosage of a therapeutic agent for delivery to an artery, wherein the dosage of therapeutic agent is arranged to be released over an administration period beginning on the date of implantation and ending between 45 days and 1 year after implantation, and wherein the therapeutic agent is calcineurin inhibitor.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is a side view of a portion of the stent of FIG. 1.

FIG. 3 is a side cross sectional view of an example of an opening in a stent showing a matrix with a therapeutic agent and polymer.

FIG. 4 is a graph of the in vivo cumulative percent of Pimecrolimus released from a stent system over time.

### DETAILED DESCRIPTION

A method for decreasing the level of restenosis following a stent placement medical intervention involves the continuous administration of a dose of an anti-restenotic agent or drug from the stent to vascular tissue in need of treatment in a controlled and extended in vivo drug release profile. It is envisioned that the vascular tissue in need of treatment is arterial tissue, specifically coronary arterial tissue. The method of extended in vivo release increases the therapeutic effectiveness of administration of a given dose of an anti-restenotic agent and more specifically an anti-inflammatory agent which reduces the inflammation involved in an angioplasty and stenting procedure.

In one example described in detail herein the agent or drug will be contained in reservoirs in the stent body prior to release. In the reservoir example, the drug will be held within the reservoirs in the stent in a drug delivery matrix comprised of the drug and a polymeric material and optionally additives to regulate the drug release. Preferably the polymeric material is a bioresorbable polymer. Although a reservoir example is described, the drug delivery stent of the present invention can include matrices fixed to a stent in a variety of manners including reservoirs, coatings, microspheres, affixed with adhesion materials or combinations thereof.

The following terms, as used herein, shall have the following meanings:

The terms “drug” and “therapeutic agent” are used interchangeably to refer to any therapeutically active substance that is delivered to a living being to produce a desired, usually beneficial, effect.

The term “matrix” or “biocompatible matrix” are used interchangeably to refer to a medium or material that, upon implantation in a subject, does not elicit a detrimental response sufficient to result in the rejection of the matrix. The matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. The matrix may be bioresorbable or non-bioresorbable.

The term “bioresorbable” refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, usually less than one year, while maintaining any requisite structural integrity in that same time period.

The term “openings” includes both through openings and recesses.

The term “pharmaceutically acceptable” refers to the characteristic of being non-toxic to a host or patient and suitable for maintaining the stability of a therapeutic agent and allowing the delivery of the therapeutic agent to target cells or tissue.

The term “polymer” refers to molecules formed from the chemical union of two or more repeating units, called monomers. Accordingly, included within the term “polymer” may be, for example, dimers, trimers, oligomers, and copolymers prepared from two or more different monomers. The polymer may be synthetic, naturally occurring or semisynthetic. In preferred form, the term “polymer” refers to molecules which typically have a Mw greater than about 3000 and preferably greater than about 10,000 and a Mw that is less than about 10 million, preferably less than about a million and more preferably less than about 200,000. Examples of polymers include but are not limited to, poly-a-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The term “primarily” with respect to directional delivery, refers to an amount greater than 50% of the total amount of therapeutic agent provided to a blood vessel.

The term “restenosis” refers to the renarrowing of an artery following an angioplasty procedure which may include stenosis following stent implantation. Restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen.

The term “anti-restenotic” refers to a drug which interferes with any one or more of the processes of restenosis to reduce the renarrowing of the lumen.

The term “substantially linear release profile” refers to a release profile defined by a plot of the cumulative drug released versus the time during which the release takes place in which the linear least squares fit of such a release profile plot has a correlation coefficient, r² (the square of the correlation coefficient of the least squares regression line), of greater than 0.92 for data time points after the first day of delivery. A substantially linear release profile is clinically significant in that it allows release of a prescribed dosage of drug at a uniform rate over an administration period. This controlled release allows a release system to stay within the toxic/therapeutic window for a particular drug over an extended administration period.

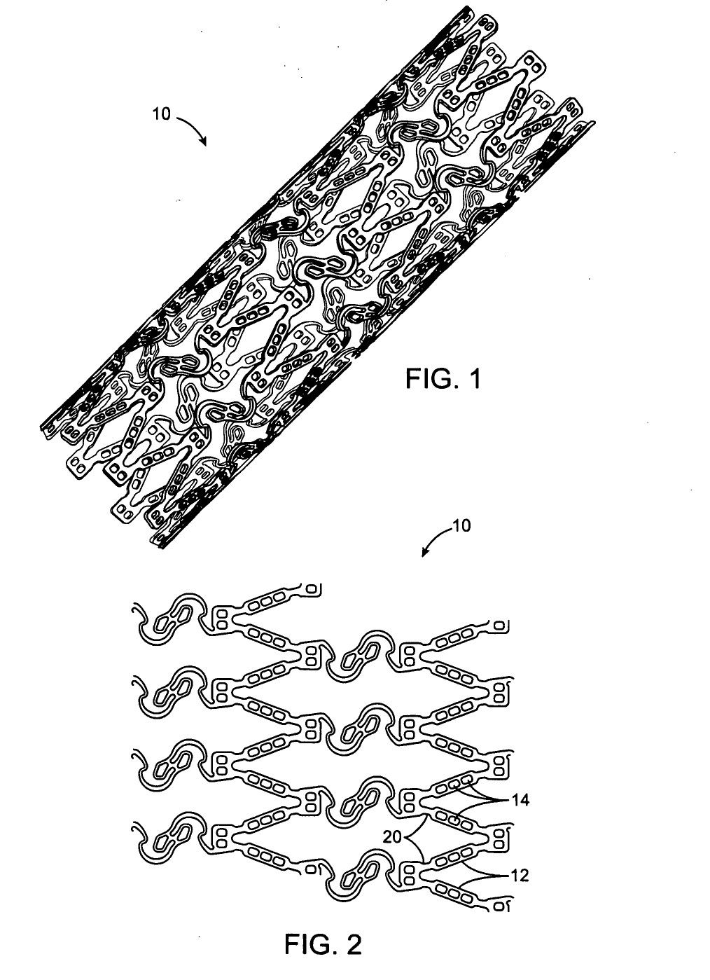

FIG. 1 illustrates one example of an implantable medical device in the form of a stent 10. FIG. 2 is an enlarged flattened view of a portion of the stent of FIG. 1 illustrating one example of a stent structure including struts 12 interconnected by ductile hinges 20. The struts 12 include openings 14 which can be non-deforming through openings containing a therapeutic agent. One example of a stent structure having non-deforming openings is shown in U.S. Pat. No. 6,562,065, which is incorporated herein by reference in its entirety.

The implantable medical devices of the present invention are configured to release at least one therapeutic agent from a matrix affixed to the implantable body. The matrix is formed such that the distribution of the agent in the polymer matrix controls the rate of elution of the agent from the matrix. The release kinetic is also controlled by the selection of the matrix, the concentration of the agent in the matrix, any additives, and any cap or rate controlling deposits.

In one embodiment, the matrix is a polymeric material which acts as a binder or carrier to hold the agent in or on the stent and/or modulate the release of the agent from the stent. The polymeric material can be a bioresorbable or a non-bioresorbable material.

The therapeutic agent containing matrix can be disposed in the stent or on surfaces of the stent in various configurations, including within volumes defined by the stent, such as openings, holes, or concave surfaces, as a reservoir of agent, or arranged in or on all or a portion of surfaces of the stent structure. When the therapeutic agent matrix is disposed within openings in the strut structure of the stent to form a reservoir, the openings may be partially or completely filled with matrix containing the therapeutic agent.

The therapeutic agent described herein which is loaded into or onto a stent is the anti-inflammatory agent Pimecrolimus. However, other anti-inflammatory agents can also be delivered according to the release profiles described herein for the treatment of restenosis.

Pimecrolimus belongs to a family of drugs called calcineurin inhibitors. Other calcineurin inhibitors include Tacrolimus, Cyclosporin A, and Ascomycin. Pimecrolimus binds to cytoplasmic proteins (FKBP 12) creating a complex that binds the calcium-dependent phosphate, calcineurin, inhibiting its ability to dephosphorylate the nuclear factor of activated T cells (NF-AT). This inhibits T cell proliferation and production, and the release of several growth factors and inflammatory cytokines (interleukin-2 [IL-2], IL-3, IL-4, interferon-y [IFN-y] and tumor necrosis factor-α [TNF-α]. Pimecrolimus also inhibits mast cell degranulation and release of pro-inflammatory mediators including histamine, cytokines, tryptase and eicosanoids.

Pimecrolimus is a hydrophobic compound used to target the inflammatory response of arterial tissue at the site of stent implantation thus reducing the stimulus for neointimal hyperplasia following stent implant. The hydrophobicity of a compound is represented by it's Log P value, where P is the octanol to water partition coefficient of the compound. Pimecrolimus has a calculated Log P value of 7.0, while Tacrolimus (Log P 5.6) has a similar high hydrophobicity and, Cyclosporin A (Log P 3.0) and Ascomycin (Log P 4.3) are not as hydrophobic.

The drug dose of Pimecrolimus is specifically tailored for localized therapy with a targeted drug release kinetic profile. This drug release kinetic profile is designed to target the immediate inflammatory response at the time of stent implantation which peaks at about 24 hours. The drug release kinetic has a bolus delivery of agent in the first 12-48 hours after implantation, followed by a controlled, extended, and preferably substantially linear, release profile during re-endothelialization of the stent/vessel wall. A total drug load of Pimecrolimus of about 100 μg to about 600 μg, preferably about 200 μg to about 400 μg is delivered from a 3 mm×16 mm stent over a period of at least 30 days, preferably at least 45 days, and more and preferably at least 45 days in vivo.

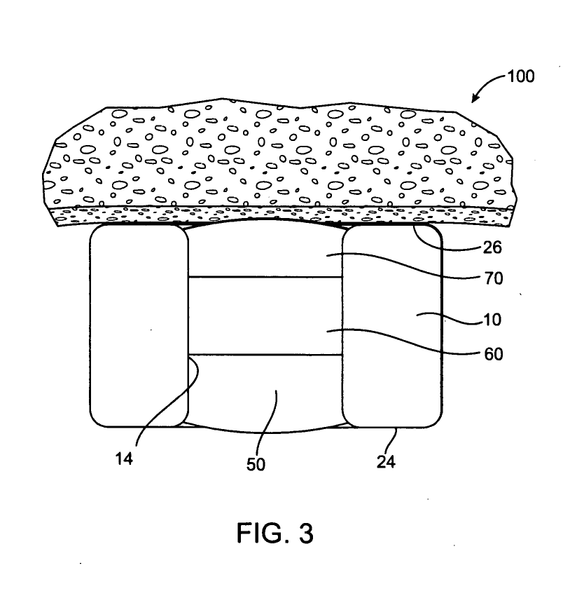

FIG. 3 is a cross section of one strut of the stent 10 and blood vessel 100 illustrating one example of an opening 14 arranged adjacent the vessel wall with a mural surface 26 abutting the vessel wall and a luminal surface 24 opposite the mural surface. The opening 14 of FIG. 3 contains an inlay formed of three regions, a luminal region 50, a middle region 60, and a mural region 70. According to one embodiment, the luminal region 50 is considered a base which is formed of matrix with little or substantially no therapeutic agent. This base 50 causes the therapeutic agent to be delivered primarily to the mural side 26 of the stent so that it is delivered directly to the artery wall. The base 50 may be formed of a material which also forms the matrix for the therapeutic agent or of a different material. Alternatively, the matrix in the base 50 can be selected to erode more slowly than the matrix containing the therapeutic agent. This can be achieved by selecting a different molecular weight of the matrix in the base 50, by different processing (i.e., annealing) of the same matrix, or by other means. A thickness of the base 50 can vary from about 5% to about 75%, preferably about 10% to 50%, of the depth of the opening 14.

The middle region 60 and the mural region 70 each include a matrix and the therapeutic agent. The agent is arranged in the matrix in a programmable manner to achieve a desired in vivo release rate and administration period which will be described in further detail below. For example the agent can be arranged in a homogeneous and even concentration throughout the middle and mural regions 60, 70, or the agent can be arranged with one or more continuous concentration gradients, one or more constant concentration areas or combinations of these.

In one example, a rapid delivery of agent in the first 12-48 hours after implantation, followed by a controlled, extended, and preferably substantially linear, release profile during re-endothelialization can be achieved with a middle region 60 formed of a combination of drug and matrix having 5-75% drug and a mural region 70 formed of a combination of a drug and matrix having a higher drug load, such as 50-100% drug. This configuration and other configurations of concentration gradients within the matrix allow the in vivo release profile to be programmed to match a particular application. In contrast, a uniform agent distribution in the matrix would result in a generally first order release profile with a large burst followed by a slower release.

The methods by which the drug can be precisely arranged within the openings 14 include a stepwise deposition processes further described in U.S. Patent Publications 2005-0010170 and 2004-0073294, both of which are incorporated herein by reference in their entirety. These stepwise deposition processes can create the regions described in FIG. 3. Although the regions have been illustrated as distinct level layers, the regions may also intermix on their margins and/or form concave, convex, or other shaped regions.

Numerous other useful arrangements of the matrix and therapeutic agent can be formed to achieve the substantially linear release, increasing release rate, extended release, and substantially complete release described herein. Each of the areas of the matrix may include one or more agents in the same or different proportions from one area to the next. The matrix may be solid, porous, or filled with other drugs or excipients. The agents may be homogeneously disposed or heterogeneously disposed in different areas of the matrix.

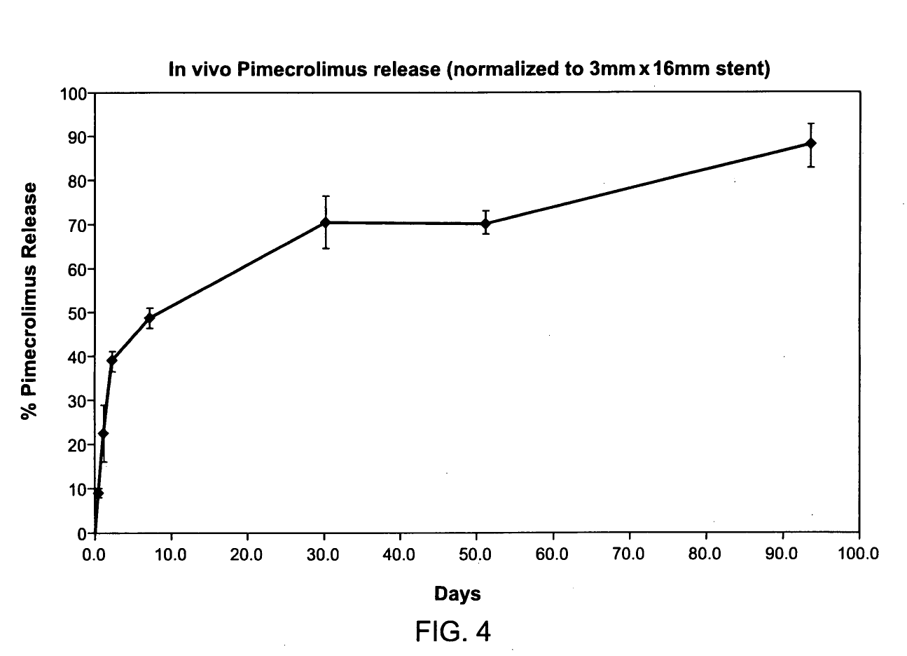

In the example of FIG. 4, a stent is cut from a cobalt chromium alloy according to the pattern shown in FIGS. 1 and 2 and Pimecrolimus is loaded in a PLGA matrix within reservoirs in the stent. The drug and matrix are arranged for directional delivery of the drug primarily to the mural side of the stent. The in vivo drug release rate is programmed by providing different concentrations of drug in different regions of the matrix similar to the regions shown in FIG. 3, with a luminal region 50 of primarily PLGA, a middle region 60 of about 75% Pimecrolimus and about 25% PLGA, and a mural region 70 of about 95% Pimecrolimus and about 5% PLGA. The reservoirs are filled, by volume, with about 20-25% base or luminal region, 40-45% middle region, and 25-30% mural region. The in vivo drug releases described herein are normalized for a 3.0 mm diameter×16 mm long expanded stent which has almost 500 reservoirs and a total drug volume of about 0.54 mm³.

When the anti-restenotic agent delivered by the method of the invention is Pimecrolimus, the total amount delivered (and loaded) is preferably between about 50 μg and about 600 μg depending on the size of the stent.

The methods of the invention will result in sustained release of substantially all the drug loaded onto the stent as well as the polymer matrix over an administration period which lasts at least 30 days and preferably no longer than 1 year.

FIG. 4 illustrates one example of an in vivo extended Pimecrolimus release profile from a bioresorbable matrix. The release profile is characterized by a moderate initial release or bolus of drug in the first 12-48 hours, followed by an extended, substantially linear release from day 2 until about 45 to 150 days, followed by a decreasing release until all the drug loaded on the stent is released between about 90 and 300 days.

The total drug load on the stents of FIG. 4 is between about 150 μg and about 400 μg normalized for a 3 mm×16 mm stent. The initial release in the first day is about 5-50%, preferably about 20-45%, of the total amount of Pimecrolimus loaded on the stent, and the initial release in the first two days is about 20-60%, preferably about 25-50%. After day two, the release rate drops to under about 15 μg per day and, preferably about 1 μg to about 10 μg per day and continues at this reduced rate for up to about 180 days. The extended phase of release after the first two days results in less than 80% of the Pimecrolimus being delivered in the first 30 days. More specifically, the 4 hour release is about 10%, the 24 hour release is about 25%, and the 2 day release is about 40%, the 8 day release is about 50%, and the 30 and 50 day releases are about 70%.

As shown in FIG. 4, the release after day two is substantially linear until less than 10% of the total drug is remaining on the stent. A dosage of about 300-350 μg on a 3 mm×16 mm size stent corresponds to about 2.0-2.4 μg/mm² of vessel surface area and about 18-22 μg/mm of vessel length. Equivalent dosages are used on stents of other sizes. When the Pimecrolimus is used in combination with another anti-restenotic drug, the dosage can be ½ of this dosage.

The initial bolus and slow extended release result in the in vivo release of not more than 90%, preferably not more than 80% of the Pimecrolimus on the stent in the first 45 days after implantation. This is followed by the complete release of the entire dose of Pimecrolimus loaded on the stent within about 1 year and preferably within about 6 months. A similar in vivo release is also used for other anti-inflammatory agents.

According to one example, the polymer is resorbed in vivo at a rate slower than the release of the drug. Therefore, substantially all of the Pimecrolimus is delivered before the polymer matrix is completely resorbed. In one embodiment the drug is completely delivered at about 1-3 months, preferably about 1-2 months, before the polymer is completely resorbed. Preferably, the polymer is completely resorbed between 45 days and 1 year from the date of implantation. The use of the resorbable polymer which completely disappears from the stent within a period of months allows an administration of antiplatelet drugs to the patient according to current procedures for drug eluting stents to be discontinued after the polymer is completely resorbed and the drug has been released. There is no non-releasable drug or polymer remaining once the stent has been in physiologic conditions for 1 year. The drug can be considered completely released or the administration can be considered complete once 95% or more of the drug which is releasable from the stent has been released.

The method of extended in vivo release of anti-inflammatory agents increases the therapeutic effectiveness of administration of a given dose of agent and reduces side effects.

While the invention has been described with respect to treatment of restenosis, other therapeutic agents may be delivered at the in vivo release profiles described for treatment of acute myocardial infarction, thrombosis, or for passivation of vulnerable plaque.

Therapeutic Agents

The present invention relates to the in vivo release kinetics involved in delivering anti-inflammatory agents including, without limitation, Pimecrolimus, salicylic acid derivatives (e.g., aspirin, sodium salicylate, choline magnesium trisalicylate, salsalate, dflunisal, salicylsalicylic acid, sulfasalazine, and olsalazine), para-amino phenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (e.g., ibuprofen, naproxen, flurbiprofen, ketoprofen, fenoprofen, and oxaprozin), anthranilic acids (e.g., mefenamic acid and meclofenamic acid), enolic acids (e.g., piroxicam, tenoxicam, phenylbutazone and oxyphenthatrazone), alkanones (e.g., nabumetone), glucocorticoids (e.g., dexamethaxone, prednisolone, and triamcinolone), pirfenidone, and tranilast.

These anti-inflammatory agents can be delivered alone or in combination. The therapeutic agents for use with the present invention which may be transmitted primarily luminally, primarily murally, or both.

Other therapeutic agents for use with the present invention may, for example, take the form of small molecules, peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense oligonucleotides, monoclonal antibodies, platelets, prions, viruses, bacteria, eukaryotic cells such as endothelial cells, stem cells, ACE inhibitors, monocyte/macrophages and vascular smooth muscle cells. Such agents can be used alone or in various combinations with one another. For instance, anti-inflammatories may be used in combination with antiproliferatives to mitigate the reaction of tissue to the antiproliferative. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the matrix. Therapeutic agents may also be radioactive isotopes or agents activated by some other form of energy such as light or ultrasonic energy, or by other circulating molecules that can be systemically administered.

Exemplary classes of therapeutic agents include antiproliferatives, antithrombins (i.e., thrombolytics), immunosuppressants, antilipid agents, anti-inflammatory agents, antineoplastics including antimetabolites, antiplatelets, angiogenic agents, anti-angiogenic agents, vitamins, antimitotics, metalloproteinase inhibitors, NO donors, nitric oxide release stimulators, anti-sclerosing agents, vasoactive agents, endothelial growth factors, beta blockers, AZ blockers, hormones, statins, insulin growth factors, antioxidants, membrane stabilizing agents, calcium antagonists (i.e., calcium channel antagonists), retinoids, anti-macrophage substances, antilymphocytes, cyclooxygenase inhibitors, immunomodulatory agents, angiotensin converting enzyme (ACE) inhibitors, anti-leukocytes, high-density lipoproteins (HDL) and derivatives, cell sensitizers to insulin, prostaglandins and derivatives, anti-TNF compounds, hypertension drugs, protein kinases, antisense oligonucleotides, cardio protectants, petidose inhibitors (increase blycolitic metabolism), endothelin receptor agonists, interleukin-6 antagonists, anti-restenotics, vasodilators, PPAR gamma agonists, and other miscellaneous compounds.

Antiproliferatives include, without limitation, paclitaxel, actinomycin D, rapamycin, everolimus, tacrolimus, Zotarolimus, cyclosporin, and Pimecrolimus.

Antithrombins include, without limitation, heparin, aspirin, sulfinpyrazone, ticlopidine, ABC1XIMAB, eptifibatide, tirofiban HCL, coumarines, plasminogen, α₂-antiplasmin, streptokinase, urokinase, bivalirudin, tissue plasminogen activator (t-PA), hirudins, hirulogs, argatroban, hydroxychloroquin, BL-3459, pyridinolcarbamate, Angiomax, and dipridamole.

Immunosuppressants include, without limitation, cyclosporine, rapamycin, tacrolimus (FK-506), everolimus, Zotarolimus, etoposide, and mitoxantrone.

Antilipid agents include, without limitation, HMG CoA reductase inhibitors, nicotinic acid, probucol, and fibric acid derivatives (e.g., clofibrate, gemfibrozil, gemfibrozil, fenofibrate, ciprofibrate, and bezafibrate).

Antineoplastics include, without limitation, nitrogen mustards (e.g., mechlorethamine, cyclophosphamide, ifosfamide, melphalan, and chlorambucil), methylnitrosoureas (e.g., streptozocin), 2-chloroethylnitrosoureas (e.g., carmustine, lomustine, semustine, and chlorozotocin), alkanesulfonic acids (e.g., busulfan), ethylenimines and methylmelamines (e.g., triethylenemelamine, thiotepa and altretamine), triazines (e.g., dacarbazine), folic acid analogs (e.g., methotrexate), pyrimidine analogs (5-fluorouracil, 5-fluorodeoxyuridine, 5-fluorodeoxyuridine monophosphate, cytosine arabinoside, 5-azacytidine, and 2′,2′-difluorodeoxycytidine), purine analogs (e.g., mercaptopurine, thioguanine, azathioprine, adenosine, pentostatin, cladribine, and erythrohydroxynonyladenine), antimitotic drugs (e.g., vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin), phenoxodiol, etoposide, and platinum coordination complexes (e.g., cisplatin and carboplatin).

Antiplatelets include, without limitation, insulin, dipyridamole, tirofiban, eptifibatide, abciximab, and ticlopidine.

Angiogenic agents include, without limitation, phospholipids, ceramides, cerebrosides, neutral lipids, triglycerides, diglycerides, monoglycerides lecithin, sphingosides, angiotensin fragments, nicotine, pyruvate thiolesters, glycerol-pyruvate esters, dihydoxyacetone-pyruvate esters and monobutyrin.

Anti-angiogenic agents include, without limitation, endostatin, angiostatin, fumagillin and ovalicin.

Vitamins include, without limitation, water-soluble vitamins (e.g., thiamin, nicotinic acid, pyridoxine, and ascorbic acid) and fat-soluble vitamins (e.g., retinal, retinoic acid, retinaldehyde, phytonadione, menaqinone, menadione, and alpha tocopherol).

Antimitotics include, without limitation, vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin.

Metalloproteinase inhibitors include, without limitation, TIMP-1, TIMP-2, TIMP-3, and SmaPI.

NO donors include, without limitation, L-arginine, amyl nitrite, glyceryl trinitrate, sodium nitroprusside, molsidomine, diazeniumdiolates, S-nitrosothiols, and mesoionic oxatriazole derivatives.

NO release stimulators include, without limitation, adenosine.

Anti-sclerosing agents include, without limitation, collagenases and halofuginone.

Vasoactive agents include, without limitation, nitric oxide, adenosine, nitroglycerine, sodium nitroprusside, hydralazine, phentolamine, methoxamine, metaraminol, ephedrine, trapadil, dipyridamole, vasoactive intestinal polypeptides (VIP), arginine, and vasopressin.

Endothelial growth factors include, without limitation, VEGF (Vascular Endothelial Growth Factor) including VEGF-121 and VEG-165, FGF (Fibroblast Growth Factor) including FGF-1 and FGF-2, HGF (Hepatocyte Growth Factor), and Ang1 (Angiopoietin 1).

Beta blockers include, without limitation, propranolol, nadolol, timolol, pindolol, labetalol, metoprolol, atenolol, esmolol, and acebutolol.

Hormones include, without limitation, progestin, insulin, the estrogens and estradiols (e.g., estradiol, estradiol valerate, estradiol cypionate, ethinyl estradiol, mestranol, quinestrol, estrond, estrone sulfate, and equilin).

Statins include, without limitation, mevastatin, lovastatin, simvastatin, pravastatin, atorvastatin, and fluvastatin.

Insulin growth factors include, without limitation, IGF-1 and IGF-2.

Antioxidants include, without limitation, vitamin A, carotenoids and vitamin E.

Membrane stabilizing agents include, without limitation, certain beta blockers such as propranolol, acebutolol, labetalol, oxprenolol, pindolol and alprenolol.

Calcium antagonists include, without limitation, amlodipine, bepridil, diltiazem, felodipine, isradipine, nicardipine, nifedipine, nimodipine and verapamil.

Retinoids include, without limitation, all-trans-retinol, all-trans-14-hydroxyretroretinol, all-trans-retinaldehyde, all-trans-retinoic acid, all-trans-3,4-didehydroretinoic acid, 9-cis-retinoic acid, 11-cis-retinal, 13-cis-retinal, and 13-cis-retinoic acid.

Anti-macrophage substances include, without limitation, NO donors.

Anti-leukocytes include, without limitation, 2-CdA, IL-1 inhibitors, anti-CD 116/CD18 monoclonal antibodies, monoclonal antibodies to VCAM, monoclonal antibodies to ICAM, and zinc protoporphyrin.

Cyclooxygenase inhibitors include, without limitation, Cox-1 inhibitors and Cox-2 inhibitors (e.g., CELEBREX® and VIOXX®).

Immunomodulatory agents include, without limitation, immunosuppressants (see above) and immunostimulants (e.g., levamisole, isoprinosine, Interferon alpha, and Interleukin-2).

ACE inhibitors include, without limitation, benazepril, captopril, enalapril, fosinopril sodium, lisinopril, quinapril, ramipril, spirapril, and 2B3 ACE inhibitors.

Cell sensitizers to insulin include, without limitation, glitazones, P PAR agonists and metformin.

Antisense oligonucleotides include, without limitation, resten-NG.

Antisense oligonucleotides include, without limitation, resten-NG.

Cardio protectants include, without limitation, VIP, pituitary adenylate cyclase-activating peptide (PACAP), apoA-I milano, amlodipine, nicorandil, cilostaxone, and thienopyridine.

Petidose inhibitors include, without limitation, omnipatrilat.

Anti-restenotics include, without limitation, include vincristine, vinblastine, actinomycin, epothilone, paclitaxel, paclitaxel derivatives (e.g., docetaxel), rapamycin, rapamycin derivatives, everolimus, tacrolimus, ZoMaxx, and Pimecrolimus.

PPAR gamma agonists include, without limitation, farglitizar, rosiglitazone, muraglitazar, pioglitazone, troglitazone, and balaglitazone.

Miscellaneous compounds include, without limitation, Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEF and PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

### EXAMPLE

The measurement of in vivo Pimecrolimus release from a stent can be performed according to the following Example. The in vivo release from other implantable medical devices can be performed in a similar manner by removal of tissue and measurement of total drug load and release kinetics by high pressure liquid chromatography (HPLC).

Stents are implanted in a porcine model and explanted at selected time points by removing the entire artery section. The expanded stents are labeled and frozen. The tissue is removed from the stent by slicing the tissue on the outside of the stent lengthwise, inverting the tissue, and removing the tissue by cutting and turning the tissue inside out. The stent may still be covered by a tough elastic membrane which is then removed by splitting the membrane and peeling it off the stent. For longer time points, there will also be a tube of tissue inside the stent. This tube is separated from the stent with tweezers, turned inside out and pulled out of the stent.

The following is the test procedure for generating the in vivo release curves for Pimecrolimus in FIG. 4.

The total drug load (TDL) of Pimecrolimus from a stent is determined by extracting all the polymer and drug from the stent in the solvent acetonitrile. The amount of Pimecrolimus in a solution sample is determined by High Pressure Liquid Chromatography (HPLC). The following conditions are used:

Analysis Column: Chromolith (100 mm×4.6 mm 3 micron RP-E)

Mobile phase: Water/Acetonitrile: 68% vol./32% vol.

Flow Rate: 1.5 mL/minute

Temperature: 50° C.

Detection wavelength: 194 nm

Injection volume: 30 μL

Retention time: 15 minutes

The TDL for the example of FIG. 4 is about 325 μg normalized to a 3 mm×16 mm stent.

The in vivo release kinetic (RK) for Pimecrolimus from a stent is determined by running the TDL for multiple explanted time points. The TDL for the explanted samples is subtracted from the TDL of an unimplanted stent to determine the amount of Pimecrolimus released at each of the explanted time points.

FIG. 4 shows that after day 2, the release profile is substantially linear with a least squares correlation coefficient (r²) of about 0.973.

While the invention has been described in detail with reference to the preferred embodiments thereof, it will be apparent to one skilled in the art that various changes and modifications can be made and equivalents employed, without departing from the present invention.

What is claimed is:

1. A method of reducing restenosis comprising:
providing a drug delivery stent having a dosage of Pimecrolimus for delivery to an artery;
implanting the stent within an artery of a patient; and
delivering Pimecrolimus from the stent in vivo over an administration period beginning on the date of implantation and ending between 45 days and 1 year after implantation.

2. The method of claim 1, wherein after the administration period no Pimecrolimus remains on the stent.

3. The method of claim 1, wherein the administration period ends between about 90 and 180 days from the date of implantation.

4. The method of claim 1, wherein the release profile of the Pimecrolimus after day two is substantially linear.

5. The method of claim 1, wherein the amount of Pimecrolimus released per day after day two is about 1 μg to about 1 μg per day delivered from a 3 mm by 16 mm expanded stent, and equivalent dosages are delivered from stents of other sizes.

6. The method of claim 1, wherein the Pimecrolimus is deposited in openings in the stent.

7. The method of claim 1, wherein the Pimecrolimus is contained in a bioresorbable matrix.

8. The method of claim 1, wherein the Pimecrolimus is contained in a polymer matrix.

9. The method of claim 8, wherein the polymer matrix is completely resorbed between 45 days and 1 year from the date of implantation.

10. The method of claim 9, wherein the step of delivering Pimecrolimus further comprises delivering substantially all the Pimecrolimus from the stent before the polymer matrix is completely resorbed.

11. The method of claim 1, wherein the Pimecrolimus is delivered primarily murally from the stent.

12. The method of claim 1, wherein the step of delivering Pimecrolimus further comprises delivering 20-60% of the total amount of Pimecrolimus loaded into the stent in the first two days.

13. The method of claim 1, wherein the step of delivering Pimecrolimus further comprises delivering 5-50% of the total amount of Pimecrolimus loaded into the stent in the first day.

14. The method of claim 1, wherein the step of delivering Pimecrolimus further comprises delivering substantially all of the Pimecrolimus loaded on the stent in no longer than 180 days.

15. The method of claim 1, wherein the step of delivering Pimecrolimus delivers not more than 80% of the Pimecrolimus in the first 30 days.

16. A stent for reducing restenosis comprising:
a drug delivery stent having initial unexpanded diameter for insertion of the stent into a coronary artery and an expanded diameter for implantation within a coronary artery, the stent having a dosage of Pimecrolimus for delivery to an artery, wherein the dosage of Pimecrolimus is arranged to be released over an administration period beginning on the date of implantation and ending between 45 days and 1 year after implantation.

17. The stent of claim 16, wherein the dosage is arranged such that substantially all the Pimecrolimus is releasable from the stent during the administration period.

18. The stent of claim 16, wherein the administration period ends between about 90 and 180 days from the date of implantation.

19. The stent of claim 16, wherein the release rate of the Pimecrolimus after day two is substantially linear.

20. The stent of claim 16, wherein the amount of Pimecrolimus released per day after day one is about 1 μg to about 10 μg per day delivered from a 3 mm by 16 mm expanded stent, and equivalent dosages are delivered from stents of other sizes.

21. The stent of claim 16, wherein the Pimecrolimus is deposited in openings in the stent.

22. The stent of claim 16, wherein the Pimecrolimus is contained in a bioresorbable matrix.

23. The stent of claim 16, wherein the Pimecrolimus is contained in a polymer matrix.

24. The stent of claim 23, wherein the polymer matrix is completely resorbed between 45 days and 1 year from the date of implantation.

25. The stent of claim 24, wherein the polymer matrix is selected to delivery substantially all the Pimecrolimus from the stent before the polymer matrix is completely resorbed.

26. The stent of claim 16, wherein the Pimecrolimus is arranged to be delivered primarily murally from the stent.

27. The stent of claim 16, wherein the Pimecrolimus is affixed to the stent such that 25-60% of the total amount of Pimecrolimus loaded into the stent is delivered in the first two days.

28. The stent of claim 16, wherein the Pimecrolimus is affixed to the stent such that 20-50% of the total amount of Pimecrolimus loaded into the stent is delivered in the first day.

29. The stent of claim 16, wherein a total dosage of Pimecrolimus delivered from a 3 mm by 16 mm expanded stent is about 150 μg to 600 μg, and equivalent dosages are delivered from stents of other sizes.

30. A stent for reducing restenosis comprising:
a drug delivery stent having initial unexpanded diameter for insertion of the stent into a coronary artery and an expanded diameter for implantation within a coronary artery, the stent having a dosage of a therapeutic agent for delivery to an artery, wherein the dosage of therapeutic agent is arranged to be released over an administration period beginning on the date of implantation and ending between 45 days and 1 year after implantation, and wherein the therapeutic agent is calcineurin inhibitor.

31. The stent of claim 30, wherein the dosage is arranged such that substantially all the therapeutic agent is releasable from the stent during the administration period.

32. The stent of claim 30, wherein the administration period ends between about 90 and 180 days from the date of implantation.

33. The stent of claim 30, wherein the release rate of the therapeutic agent after day two is substantially linear.

34. The stent of claim 30, wherein the therapeutic agent is deposited in openings in the stent.

35. The stent of claim 30, wherein the therapeutic agent is contained in a bioresorbable matrix.

36. The stent of claim 30, wherein the therapeutic agent is contained in a polymer matrix.

37. The stent of claim 36, wherein the polymer matrix is completely resorbed between 45 days and 1 year from the date of implantation.

38. The stent of claim 37, wherein the polymer matrix is selected to delivery substantially all the therapeutic agent from the stent before the polymer matrix is completely resorbed.

39. The stent of claim 30, wherein the therapeutic agent is arranged to be delivered primarily murally from the stent.

40. The stent of claim 30, wherein the therapeutic agent is affixed to the stent such that 25-60% of the total amount of therapeutic agent loaded into the stent is delivered in the first two days.

41. The stent of claim 30, wherein the therapeutic agent is affixed to the stent such that 20-50% of the total amount of therapeutic agent loaded into the stent is delivered in the first day.
